SEQUENCE LISTING (17 sequences)

SEQ ID NO: 1 - protein, 7 aa, synthetic construct
GFSLTRN

SEQ ID NO: 2 - protein, 5 aa, synthetic construct
WGDGT

SEQ ID NO: 3 - protein, 7 aa, synthetic construct
GGSREAF

SEQ ID NO: 4 - protein, 11 aa, synthetic construct
RASQSISDYLH

SEQ ID NO: 5 - protein, 7 aa, synthetic construct
YASQSIS

SEQ ID NO: 6 - protein, 9 aa, synthetic construct
QNGHSFPLT

SEQ ID NO: 7 - protein, 7 aa, synthetic construct
GYTFTSY

SEQ ID NO: 8 - protein, 6 aa, synthetic construct
YPGNVN

SEQ ID NO: 9 - protein, 11 aa, synthetic construct
GSRYGYWYFDV

SEQ ID NO: 10 - protein, 16 aa, synthetic construct
RSSQSIVHSNGNTYLE

SEQ ID NO: 11 - protein, 7 aa, synthetic construct
KVSNRFS

SEQ ID NO: 12 - protein, 9 aa, synthetic construct
FQGSHVPLT

SEQ ID NO: 13 - protein, 115 aa, synthetic construct
QVQLKESGPGLVAPSQSLSITCTVSGFSLTRNGVSWVRQPPGKGLEWLGVIWGDGTTNYHSALISRLSISKDNSKSQVFLKLNSLRTDDTATYYCAKGGSREAFWGQGTLVTVSA

SEQ ID NO: 14 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 15 - protein, 120 aa, synthetic construct
QVQLQQSGPELVKPGASVRISCKASGYTFTSYYIQWMKQRPGQGLEWIGWIYPGNVNTKYNEKFKGKATLTADKSSSTAYMHLNSLTSEDSAAYFCARGSRYGYWYFDVWGAGTTVTVSS

SEQ ID NO: 16 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 17 - protein, 135 aa, synthetic construct
SIDVKYIGVKSAYVSYDVQKRTIYLNITNTLNITNNNYYSVEVENITAQVQFSKTVIGKARLNNITIIGPLDMKQIDYTVPTVIAEEMSYMYDFCTLISIKVHNIVLMMQVTVTTTYFGHSEQISQERYQYVDCG

(19) United States
(12) Patent Application Publication
(10) Pub. No.: US 2026/0347463 A1
(43) Pub. Date: Nov. 5, 2026

(54) TMEM106B(120-254) AGGREGATE-BINDING ANTIBODY

(71) Applicant: The Trustees of Columbia University in the City of New York, New York, NY (US)

(72) Inventors: Anthony William Paul FITZPATRICK, New York, NY (US); Junqiang YE, New York, NY (US); Susan L. BRENNER-MORTON, New York, NY (US); Carolyn LEE, New York, NY (US)

(21) Appl. No.: 19/678,901

(22) Filed: May 15, 2026

Related U.S. Application Data
(63) Continuation of application No. PCT/US2024/057437, filed on Nov. 26, 2024.
(60) Provisional application No. 63/602,923, filed on Nov. 27, 2023.
(60) Provisional application No. 63/647,206, filed on May 14, 2024.

(51) Int. Cl. C07K 16/28; A61K 38/00; A61P 25/28; C12N 15/62

(52) U.S. Cl. CPC C07K 16/28; A61K 38/00; A61P 25/28; C12N 15/62; C07K 2317/14; C07K 2317/24; C07K 2317/52; C07K 2317/565; C07K 2319/00

(57) ABSTRACT

Anti-TMEM106B(120-254) aggregate antibodies, antibody fragments or fusion proteins are provided and methods of treating dementias, including Frontotemporal Lobar Degeneration, using anti-TMEM106B(120-254) aggregate antibodies, antibody fragments or fusion proteins.

### CROSS-REFERENCE TO RELATED APPLICATIONS

This application is a continuation of PCT International Application No. PCT/US2024/057437, filed Nov. 26, 2024, which claims benefit of U.S. Provisional Application No. 63/602,923, filed Nov. 27, 2023, and U.S. Provisional Application No. 63/647,206, filed May 14, 2024, the contents of each of which are hereby incorporated by reference.

### STATEMENT OF GOVERNMENTAL INTEREST

This invention was made with government support under grant numbers NS110438 and NS110435 awarded by the National Institutes of Health. The government has certain rights in the invention.

### REFERENCE TO SEQUENCE LISTING

This application incorporates-by-reference nucleotide sequences which are present in the file named “93597-7729_92183-A-PCT-A_Substitute_Sequence_Listing_AWG.xml”, which is 16,340 bytes in size, and which was created on Jul. 10, 2026, in the IBM-PC machine format, having an operating system compatibility with MS-Windows, which is contained in the xml file filed Jul. 10, 2026 as part of this application.

### BACKGROUND

Recently, it was discovered that a C-terminal fragment of Transmembrane Protein 106B, TMEM106B(120-254), aggregates into amyloid fibrils in multiple neurodegenerative diseases. To date, an antibody that binds exclusively to TMEM106B(120-254) aggregates does not exist. Such an antibody would be useful for treating neurodegenerative diseases which implicate TMEM106B(120-254) aggregates including, for example, frontotemporal lobar degeneration.

### SUMMARY

An antibody, antigen-binding fragment thereof, or fusion protein, which binds TMEM106B(120-254) aggregate, comprising:

a) a heavy chain comprising:
CDR1 having the sequence
(SEQ ID NO: 1)
GFSLTRN;
CDR2 having the sequence
(SEQ ID NO: 2)
WGDGT;
CDR3 having the sequence
(SEQ ID NO: 3)
GGSREAF;
and
a light chain comprising:
CDR1 having the sequence
(SEQ ID NO: 4)
RASQSISDYLH;
CDR2 having the sequence
(SEQ ID NO: 5)
YASQSIS;
CDR3 having the sequence
(SEQ ID NO: 6)
QNGHSFPLT;
or
b) a heavy chain comprising:
CDR1 having the sequence
(SEQ ID NO: 7)
GYTFTSY;
CDR2 having the sequence
(SEQ ID NO: 8)
YPGNVN;
CDR3 having the sequence
(SEQ ID NO: 9)
GSRYGYWYFDV;
and a light chain comprising:
CDR1 having the sequence
(SEQ ID NO: 10)
RSSQSIVHSNGNTYLE;
CDR2 having the sequence
(SEQ ID NO: 11)
KVSNRFS;
CDR3 having the sequence
(SEQ ID NO: 12)
FQGSHVPLT.

An antibody, antigen-binding fragment thereof, or fusion protein, which binds TMEM106B(120-254) aggregate, comprising complementary-determining regions CDR1 through CDR3 of a heavy chain and CDR1 through CDR3 of a light chain, wherein one or more of CDR1 through CDR3 of the heavy chain, or light chain, has 85% or greater identity with, but not 100% identity with, the CDR1 through CDR3 sequences of the heavy chain, or light chain, respectively, set forth as follows:

c) a heavy chain comprising:
CDR1 having the sequence
(SEQ ID NO: 1)
GFSLTRN;
CDR2 having the sequence
(SEQ ID NO: 2)
WGDGT;
CDR3 having the sequence
(SEQ ID NO: 3)
GGSREAF;
and
a light chain comprising:
CDR1 having the sequence
(SEQ ID NO: 4)
RASQSISDYLH;
CDR2 having the sequence
(SEQ ID NO: 5)
YASQSIS;
CDR3 having the sequence
(SEQ ID NO: 6)
QNGHSFPLT;
or
d) a heavy chain comprising:
CDR1 having the sequence
(SEQ ID NO: 7)
GYTFTSY;
CDR2 having the sequence
(SEQ ID NO: 8)
YPGNVN;
CDR3 having the sequence
(SEQ ID NO: 9)
GSRYGYWYFDV;
and
a light chain comprising:
CDR1 having the sequence
(SEQ ID NO: 10)
RSSQSIVHSNGNTYLE;
CDR2 having the sequence
(SEQ ID NO: 11)
KVSNRFS;
CDR3 having the sequence
(SEQ ID NO: 12)
FQGSHVPLT.

A pharmaceutical composition comprising the antibody, antigen-binding fragment thereof, or fusion protein, which binds TMEM106B(120-254) aggregate described herein and a carrier.

A method of treating or reducing development of Frontotemporal Lobar Degeneration, Lewy Body Dementia, ALS or Alzheimer's disease, comprising administering to a subject having the Frontotemporal Lobar Degeneration, Lewy Body Dementia, ALS or Alzheimer's disease an amount of an antibody which binds TMEM106B(120-254) aggregate, or TMEM106B(120-254) aggregate binding fragment thereof or fusion protein described herein, effective to reduce development of or treat Frontotemporal Lobar Degeneration, Lewy Body Dementia, ALS or Alzheimer's disease.

A method of treating or reducing development of a neurodegenerative disorder, comprising administering to a subject having the neurodegenerative disorder an amount of an antibody which binds TMEM106B(120-254) aggregate, or TMEM106B(120-254) aggregate binding fragment thereof or fusion protein described herein, effective to reduce development of or treat a neurodegenerative disorder.

A method of reducing development of, or accumulation of, a TMEM106B(120-254) aggregate, comprising administering to a subject having a TMEM106B(120-254) aggregate or susceptible thereto an amount of an antibody which binds TMEM106B(120-254) aggregate, or TMEM106B(120-254) aggregate binding fragment thereof or fusion protein described herein, effective to reduce development of, or accumulation of, a TMEM106B(120-254) aggregate.

In embodiments, the methods, further comprise receiving identification of the subject, or identifying the subject, as having, or being susceptible to, a TMEM106B(120-254) aggregate prior to administration of the treatment.

A nucleic acid encoding a heavy chain as described herein.

A nucleic acid encoding a light chain as described herein.

A hybridoma comprising a nucleic acid as described herein.

### BRIEF DESCRIPTION OF THE DRAWINGS

FIG. 1: Diagram of IgG sequences (VH SEQ ID NO:13; VL SEQ ID NO:14). Underlined residues are CDR1-3, consecutively; and of IgM sequences (VH SEQ ID NO:15; VL SEQ ID NO:16). Underlined residues are CDR1-3, consecutively.

FIG. 2: Generation of monoclonal antibodies FL1 and FL2 to TMEM106B Fibrils; One mouse was immunized IP with 7.5 μg of insoluble TMEM106B emulsified in freund's adjuvant on three occasions. The mouse rested 4.5 weeks, was boosted on with fibrils in PBS (w/o adjuvant), and was later sacrificed. The spleen was removed and frozen as a cell suspension in DMSO containing freezing media at −80 °C. Later, the frozen spleen cells were thawed and immediately fused with a parental NS-1 cell line grown to log phase. Supernatants from the fused cells were screened by ELISA on TMEM106B C-terminal peptide (100 μL/well at 20 μg/mL). FL1 and FL2 went through 2 rounds of single cell cloning. FL1 was isotyped as IgG2b and FL2 was isotyped as IgM.

FIG. 3: Immunoblotting using TMEM106B peptides to map the epitopes of FL1 and FL2 antibodies. Results indicate that FL1 (IgG) and FL2 (IgM) bind to the N-terminal region of the fibril-forming fragment, TMEM106B(120-254).

FIG. 4: Normalized Thioflavin T fluorescence intensity of an aggregation prone fragment of TMEM106B (blue curve—upper) and in the presence of FL1 antibody (red curve—lower). Protein concentration is 5 μM in 50 mM sodium phosphate, 150 mM sodium chloride, 1.25% DMSO at pH 7.4 heated to 37 °C (blue curve) and in the presence of 1:20 molar equivalent of FL1 antibody at a concentration of 0.25 μM (red curve). Final concentration of Thioflavin T is 40 μM. Kinetic profiles of aggregation of TMEM106B alone and in the presence of the IgG antibody. The antibody significantly slows aggregation at a sub-stoichiometric ratio (1:20 molarity).

FIG. 5: Normalized Thioflavin T fluorescence intensity of an aggregation prone fragment of TMEM106B (blue curve) and in the presence of FL2 antibody (red curve). Protein concentration is 5 μM in 50 mM sodium phosphate, 150 mM sodium chloride, 1.25% DMSO at pH 7.4 heated to 37 °C (blue curve) and in the presence of FL1 antibody at a final concentration of 0.25 μM (red curve). Final concentration of Thioflavin T is 40 μM.

### DETAILED DESCRIPTION

An antibody, antigen-binding fragment thereof, or fusion protein, which binds TMEM106B(120-254) aggregate, comprising:

a) a heavy chain comprising:
CDR1 having the sequence
(SEQ ID NO: 1)
GFSLTRN;
CDR2 having the sequence
(SEQ ID NO: 2)
WGDGT;
CDR3 having the sequence
(SEQ ID NO: 3)
GGSREAF;
and
a light chain comprising:
CDR1 having the sequence
(SEQ ID NO: 4)
RASQSISDYLH;
CDR2 having the sequence
(SEQ ID NO: 5)
YASQSIS;
CDR3 having the sequence
(SEQ ID NO: 6)
QNGHSFPLT;
or
b) a heavy chain comprising:
CDR1 having the sequence
(SEQ ID NO: 7)
GYTFTSY;
CDR2 having the sequence
(SEQ ID NO: 8)
YPGNVN;
CDR3 having the sequence
(SEQ ID NO: 9)
GSRYGYWYFDV;
and
a light chain comprising:
CDR1 having the sequence
(SEQ ID NO: 10)
RSSQSIVHSNGNTYLE;
CDR2 having the sequence
(SEQ ID NO: 11)
KVSNRFS;
CDR3 having the sequence
(SEQ ID NO: 12)
FQGSHVPLT.

In embodiments of the antibody, fragment thereof, or fusion protein, the heavy chain comprises:

CDR1 having the sequence
(SEQ ID NO: 1)
GFSLTRN;
CDR2 having the sequence
(SEQ ID NO: 2)
WGDGT;
CDR3 having the sequence
(SEQ ID NO: 3)
GGSREAF;

and the light chain comprises:

CDR1 having the sequence
(SEQ ID NO: 4)
RASQSISDYLH;
CDR2 having the sequence
(SEQ ID NO: 5)
YASQSIS;
CDR3 having the sequence
(SEQ ID NO: 6)
QNGHSFPLT.

In embodiments of the antibody, fragment thereof, or fusion protein the heavy chain comprises:

CDR1 having the sequence
(SEQ ID NO: 7)
GYTFTSY;
CDR2 having the sequence
(SEQ ID NO: 8)
YPGNVN;
CDR3 having the sequence
(SEQ ID NO: 9)
GSRYGYWYFDV;

and the light chain comprises:

CDR1 having the sequence
(SEQ ID NO: 10)
RSSQSIVHSNGNTYLE;
CDR2 having the sequence
(SEQ ID NO: 11)
KVSNRFS;
CDR3 having the sequence
(SEQ ID NO: 12)
FQGSHVPLT.

In embodiments of the antibody, fragment thereof, or fusion protein the antibody is a monoclonal antibody.

In embodiments, the antibody, fragment thereof, or fusion protein comprises framework regions of a light chain and/or a heavy chain which are human framework regions, or have 85% or more sequence identity thereto.

In embodiments of the antibody, fragment thereof, or fusion protein, the framework regions of the light chain and/or the heavy chain are human framework regions.

In embodiments of the antibody, fragment thereof, or fusion protein, the antibody is a humanized antibody.

In embodiments of the antibody, fragment thereof, or fusion protein, the antibody or antigen-binding fragment thereof has a human sequence Fc region.

In embodiments of the antibody, fragment thereof, or fusion protein, the antibody or fragment thereof is chimeric.

In embodiments of the antibody, fragment thereof, or fusion protein, the antibody is a monospecific antibody comprising two heavy chains of identical sequence and two light chains of identical sequence.

In embodiments of the antibody, fragment thereof, or fusion protein, the antibody is a bispecific antibody comprising (i) a heavy chain and a light chain as set forth in a) or b) described hereinabove, and (ii) a heavy chain and a light chain as set forth in a) or b) described hereinabove, wherein the heavy chain and a light chain of (i) are different in sequence from the heavy chain and a light chain of (ii).

In embodiments of the antibody, fragment thereof, or fusion protein, a heavy chain of the antibody comprises

(SEQ ID NO: 13)
QVQLKESGPGLVAPSQSLSITCTVSGFSLTRNGVSWVRQPPGKGLEWLG
VIWGDGTTNYHSALISRLSISKDNSKSQVFLKLNSLRTDDTATYYCAKG
GSREAFWGQGTLVTVSA.

In embodiments of the antibody, fragment thereof, or fusion protein, a heavy chain of the antibody comprises

(SEQ ID NO: 15)
QVQLQQSGPELVKPGASVRISCKASGYTFTSYYIQWMKQRPGQGLEWIG
WIYPGNVNTKYNEKFKGKATLTADKSSSTAYMHLNSLTSEDSAAYFCAR
GSRYGYWYFDVWGAGTTVTVSS.

In embodiments of the antibody, fragment thereof, or fusion protein, a light chain of the antibody comprises

(SEQ ID NO: 14)
DIVMTQSPATLSVTPGDRVSLSCRASQSISDYLHWYQQKSHESPRLLIK
YASQSISGIPSRFSGSGSGSDFTLSINSVEPEDVGVYYCQNGHSFPLTF
GAGTKLELK.

In embodiments of the antibody, fragment thereof, or fusion protein, a light chain of the antibody comprises

(SEQ ID NO: 16)
DVLMTQTPLSLPVSLGDQASISCRSSQSIVHSNGNTYLEWYLQKPGQSP
KLLIYKVSNRFSGVPDRFSGSGSGTDFTLKISRVEAEDLGVYYCFQGSH
VPLTFGAGTKLELK.

In embodiments of the antibody, fragment thereof, or fusion protein, the antibody is an IgG.

In embodiments of the antibody, fragment thereof, or fusion protein, the antibody is an IgM.

In embodiments of the antibody is a bispecific antibody. In embodiments the bispecific antibody is a KiH, a κλ-body, or a CrossMab.

In embodiments of the antibody, fragment thereof, or fusion protein, the antibody is an IgG2b, IgG1(λ) or an IgG4(λ).

In embodiments, the fragment is a TMEM106B(120-254) aggregate binding fragment. In embodiments the TMEM106B(120-254) aggregate binding fragment is an Fab fragment, an Fab′ fragment, or an F(ab)′ fragment.

In embodiments, the fusion protein binds a TMEM106B(120-254) aggregate and is a single chain variable fragment (scFv).

In embodiments, the fusion protein is a BiTE (bispecifc T-cell engager) or other tandem scFv, a chemically-linked F(ab′)₂, a diabody, an IgG-scFv, a TandAb (tandem diabody), a DVD-Ig (dual variable domain immunoglobulin), or a DART (dual-affinity retargeting molecule).

An antibody, antigen-binding fragment thereof, or fusion protein, which binds TMEM106B(120-254) aggregate, comprising complementary-determining regions CDR1 through CDR3 of a heavy chain and CDR1 through CDR3 of a light chain, wherein one or more of CDR1 through CDR3 of the heavy chain, or light chain, has 85% or greater identity with, but not 100% identity with, the CDR1 through CDR3 sequences of the heavy chain, or light chain, respectively, set forth as follows:

c)
a heavy chain comprising:
CDR1 having the sequence
(SEQ ID NO: 1)
GFSLTRN;
CDR2 having the sequence
(SEQ ID NO: 2)
WGDGT;
CDR3 having the sequence
(SEQ ID NO: 3)
GGSREAF;
and
a light chain comprising:
CDR1 having the sequence
(SEQ ID NO: 4)
RASQSISDYLH;
CDR2 having the sequence
(SEQ ID NO: 5)
YASQSIS;
CDR3 having the sequence
(SEQ ID NO: 6)
QNGHSFPLT;
or
d)
a heavy chain comprising:
CDR1 having the sequence
(SEQ ID NO: 7)
GYTFTSY;
CDR2 having the sequence
(SEQ ID NO: 8)
YPGNVN;
CDR3 having the sequence
(SEQ ID NO: 9)
GSRYGYWYFDV;
and
a light chain comprising:
CDR1 having the sequence
(SEQ ID NO: 10)
RSSQSIVHSNGNTYLE;
CDR2 having the sequence
(SEQ ID NO: 11)
KVSNRFS;
CDR3 having the sequence
(SEQ ID NO: 12)
FQGSHVPLT.

A pharmaceutical composition comprising the antibody, antigen-binding fragment thereof, or fusion protein, which binds TMEM106B(120-254) aggregate described herein and a carrier.

A method of treating or reducing development of Frontotemporal Lobar Degeneration, Lewy Body Dementia, ALS or Alzheimer's disease, comprising administering to a subject having the Frontotemporal Lobar Degeneration, Lewy Body Dementia, ALS or Alzheimer's disease an amount of an antibody which binds TMEM106B(120-254) aggregate, or TMEM106B(120-254) aggregate binding fragment thereof or fusion protein described herein, effective to reduce development of or treat Frontotemporal Lobar Degeneration, Lewy Body Dementia, ALS or Alzheimer's disease.

A method of treating or reducing development of a neurodegenerative disorder, comprising administering to a subject having the neurodegenerative disorder an amount of an antibody which binds TMEM106B(120-254) aggregate, or TMEM106B(120-254) aggregate binding fragment thereof or fusion protein described herein, effective to reduce development of or treat a neurodegenerative disorder.

A method of reducing development of, or accumulation of, a TMEM106B(120-254) aggregate, comprising administering to a subject having a TMEM106B(120-254) aggregate or susceptible thereto an amount of an antibody which binds TMEM106B(120-254) aggregate, or TMEM106B(120-254) aggregate binding fragment thereof or fusion protein described herein, effective to reduce development of, or accumulation of, a TMEM106B(120-254) aggregate.

In embodiments, the methods, further comprise receiving identification of the subject, or identifying the subject, as having, or being susceptible to, a TMEM106B(120-254) aggregate prior to administration of the treatment.

In embodiments, the subject is human.

A nucleic acid encoding a heavy chain as described herein.

A nucleic acid encoding a light chain as described herein.

In embodiments, the nucleic acid is an expression vector. In an embodiment, the nucleic acid is a cDNA.

A hybridoma comprising a nucleic acid as described herein.

In embodiments, the anti-TMEM106B(120-254) aggregate antibody or fragment thereof, comprises (i) a VH framework comprising the framework sequence of human germline IGHV1-2*02, IGHV1-2*04, IGHV1-2*05, IGHV1-18*04, IGHV1-69-2*01, IGHV1-46*01, IGHD5-12*01, IGHD5-24*01, IGHD6-25*01, IGHJ3*01, IGHJ4*01, IGHJ4*03, IGHJ6*01, IGHJ6*02 and/or (ii) a VL framework comprising the framework sequence of human germline IGKV1-13*02, IGKV1-27*01, IGKV3-7*02, IGKV4-1*01, IGKV1D-13*02, IGKV3D-7*01, IGKJ1*01, IGKJ2*01, IGKJ4*01, IGKJ4*02. In embodiments, the anti-TMEM106B(120-254) aggregate antibody or fragment thereof, comprises an optimized version of, having less than 100% sequence identity with, a (i) a VH framework comprising the framework sequence of human germline IGHV1-2*02, IGHV1-2*04, IGHV1-2*05, IGHV1-18*04, IGHV1-69-2*01, IGHV1-46*01, IGHD5-12*01, IGHD5-24*01, IGHD6-25*01, IGHJ3*01, IGHJ4*01, IGHJ4*03, IGHJ6*01, IGHJ6*02 and/or (ii) a VL framework comprising the framework sequence of human germline IGKV1-13*02, IGKV1-27*01, IGKV3-7*02, IGKV4-1*01, IGKV1D-13*02, IGKV3D-7*01, IGKJ1*01, IGKJ2*01, IGKJ4*01, IGKJ4*02.

Fragments of antibodies can be prepared, for example, by cleaving an intact antibody or by recombinant means. See generally, Fundamental Immunology, Ch. 7 (Paul, W., ed., 2nd ed. Raven Press, N.Y. (1989), hereby incorporated by reference in its entirety). Antigen-binding fragments may be produced by recombinant DNA techniques or by enzymatic or chemical cleavage of intact antibodies or by molecular biology techniques. In some embodiments, a fragment is an Fab, Fab′, F(ab′)2, Fd, Fv, complementarity determining region (CDR) fragment, single-chain antibody (scFv), (a variable domain light chain (VL) and a variable domain heavy chain (VH) linked via a peptide linker. In an embodiment, the scFv comprises a variable domain framework sequence having a sequence identical to a human variable domain FR1, FR2, FR3 or FR4. In an embodiment, the scFv comprises a linker peptide from 5 to 30 amino acid residues long. In an embodiment, the scFv comprises a linker peptide comprising one or more of glycine, serine and threonine residues.

In embodiments, a linker of the scFv is 10-25 amino acids in length. In an embodiment the peptide linker comprises glycine, serine and/or threonine residues. (For example, see Bird et al., Science, 242: 423-426 (1988) and Huston et al., Proc. Natl. Acad. Sci. USA, 85:5879-5883 (1988) each of which are hereby incorporated by reference in their entirety), or a polypeptide that contains at least a portion of an antibody that is sufficient to confer specific antigen binding on the polypeptide, including a diabody. From N-terminus to C-terminus, both the mature light and heavy chain variable domains comprise the regions FR1, CDR1, FR2, CDR2, FR3, CDR3 and FR4. The assignment of amino acids to each domain can be in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987), or Chothia et al., Nature 342:878-883 (1989), each of which are hereby incorporated by reference in their entirety).

The term “monoclonal antibody” as used herein refers to an antibody member of a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In contrast to polyclonal antibody preparations, which typically include different antibodies directed against different determinants (epitopes), each monoclonal antibody of a monospecific monoclonal antibody preparation is directed against a single determinant on an antigen. A bispecific antibody, recognizing two antigens, in one embodiment, can be manufactured from two monoclonal antibodies. In addition to their specificity, monoclonal antibody preparations are advantageous in that they are typically uncontaminated by other immunoglobulins. Thus, an identified monoclonal antibody can be produced by non-hybridoma techniques, e.g., by appropriate recombinant means once the sequence thereof is identified.

In an embodiment of the inventions described herein, the antibody is isolated. As used herein, the term “isolated antibody” refers to an antibody that by virtue of its origin or source of derivation has one, two, three or four of the following: (1) is not associated with naturally associated components that accompany it in its native state, (2) is free of other proteins from the same species, (3) is expressed by a cell from a different species, and (4) does not occur in nature absent the hand of man.

In an embodiment the antibody is humanized. “Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region (HVR) (or CDR) of the recipient are replaced by residues from a HVR (or CDR) of a non-human species (donor antibody) such as mouse, rat, rabbit, or nonhuman primate having the desired specificity, affinity, and/or capacity. In an embodiment, the antibody has 1, 2, 3, 4, 5, or all 6 CDR1-3 of both the heavy and light chain of the antibodies described herein. In a preferred embodiment, framework (FR) residues of the murine mAb are replaced with corresponding human immunoglobulin variable domain framework (FR) residues. These may be modified further in embodiments to further refine antibody performance. Furthermore, in a specific embodiment, humanized antibodies may comprise residues that are not found in the recipient antibody or in the donor antibody. In an embodiment, the humanized antibodies do not comprise residues that are not found in the recipient antibody or in the donor antibody. In general, a humanized antibody will comprise substantially all of at least one, and typically two, variable domains, in which all, or in embodiments substantially all, of the hypervariable loops correspond to those of a non-human immunoglobulin, and all, or in embodiments substantially all, of the FRs are those of a human immunoglobulin sequence. The humanized antibody optionally will also comprise at least a portion of an immunoglobulin constant region (Fc), typically that of a human immunoglobulin. See, e.g., Jones et al., Nature 321:522-525 (1986); Riechmann et al., Nature 332:323-329 (1988); Presta, Curr. Op. Struct. Biol. 2:593-596 (1992); Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409, the contents of each of which references and patents are hereby incorporated by reference in their entirety. In one embodiment where the humanized antibodies do comprise residues that are not found in the recipient antibody or in the donor antibody, the Fc regions of the antibodies are modified as described in WO 99/58572, the content of which is hereby incorporated by reference in its entirety.

Techniques to humanize a monoclonal antibody are well known. Some are described in, for example, U.S. Pat. Nos. 4,816,567; 5,807,715; 5,866,692; 6,331,415; 5,530,101; 5,693,761; 5,693,762; 5,585,089; and 6,180,370, the content of each of which is hereby incorporated by reference in its entirety. A number of “humanized” antibody molecules comprising an antigen-binding site derived from a non-human immunoglobulin have been described, including antibodies having rodent or modified rodent V regions and their associated complementarity determining regions (CDRs) fused to human constant domains. See, for example, Winter et al. Nature 349: 293-299 (1991), Lobuglio et al. Proc. Nat. Acad. Sci. USA 86: 4220-4224 (1989), Shaw et al. J. Immunol. 138: 4534-4538 (1987), and Brown et al. Cancer Res. 47: 3577-3583 (1987), the content of each of which is hereby incorporated by reference in its entirety. Other references describe rodent hypervariable regions or CDRs grafted into a human supporting framework region (FR) prior to fusion with an appropriate human antibody constant domain. See, for example, Riechmann et al. Nature 332: 323-327 (1988), Verhoeyen et al. Science 239: 1534-1536 (1988), and Jones et al. Nature 321: 522-525 (1986), the content of each of which is hereby incorporated by reference in its entirety. Another reference describes rodent CDRs supported by recombinantly veneered rodent framework regions—European Patent Publication No. 0519596 (incorporated by reference in its entirety). These “humanized” molecules are designed to minimize unwanted immunological response toward rodent anti-human antibody molecules which limits the duration and effectiveness of therapeutic applications of those moieties in human recipients. The antibody constant region can be engineered such that it is immunologically inert (e.g., does not trigger complement lysis). See, e.g. PCT Publication No. WO99/58572; UK Patent Application No. 9809951.8. Other methods of humanizing antibodies that may also be utilized are disclosed by Daugherty et al., Nucl. Acids Res. 19: 2471-2476 (1991) and in U.S. Pat. Nos. 6,180,377; 6,054,297; 5,997,867; 5,866,692; 6,210,671; and 6,350,861; and in PCT Publication No. WO 01/27160 (each incorporated by reference in their entirety).

Other forms of humanized antibodies have one or more, or all, CDRs (CDR L1, CDR L2, CDR L3, CDR H1, CDR H2, or CDR H3) which are altered with respect to the original antibody, which are also termed one or more CDRs “derived from” one or more CDRs from the original antibody.

In embodiments, the antibodies or fragments herein can be produced recombinantly, for example antibodies expressed using a recombinant expression vector transfected into a host cell, antibodies isolated from a recombinant, combinatorial human antibody library, antibodies isolated from an animal (e.g., a mouse) that is transgenic for human immunoglobulin genes.

The term “K_{d}”, as used herein, is intended to refer to the dissociation constant of an antibody-antigen interaction. One way of determining the K_{d} or binding affinity of antibodies to the TMEM106B(120-254) aggregate can be by measuring binding affinity of monofunctional Fab fragments of the antibody. (The affinity constant is the inverted dissociation constant). To obtain monofunctional Fab fragments, an antibody (for example, IgG) can be cleaved with papain or expressed recombinantly. The affinity of a fragment of an antibody can be determined, for example, by surface plasmon resonance (BIAcore3000™ surface plasmon resonance (SPR) system, BIAcore Inc., Piscataway N.J.). CM5 chips can be activated with N-ethyl-N′-(3-dimethylaminopropyl)-carbodiinide hydrochloride (EDC) and N-hydroxysuccinimide (NHS) according to the supplier's instructions. The antigen can be diluted into 10 mM sodium acetate pH 4.0 and injected over the activated chip at a concentration of 0.005 mg/mL. Using variable flow time across the individual chip channels, two ranges of antigen density can be achieved: 100-200 response units (RU) for detailed kinetic studies and 500-600 RU for screening assays. Serial dilutions (0.1-10× estimated K_{d}) of purified Fab samples are injected for 1 min at 100 microliters/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data to a 1:1 Langmuir binding model (Karlsson, R. Roos, H. Fagerstam, L. Petersson, B. (1994). Methods Enzymology 6. 99-110, the content of which is hereby incorporated in its entirety) using the BIA evaluation program. Equilibrium dissociation constant (K_{d}) values are calculated as k_{off}/kₒₙ. This protocol is suitable for use in determining binding affinity of an antibody or fragment to any antigen. Other protocols known in the art may also be used. For example, ELISA.

In embodiments, the antibody or antigen-binding fragment thereof or fusion protein binds to TMEM106B(120-254) aggregate with an affinity of 100.0 nM K_{D} or stronger.

In embodiments, the antibody or antigen-binding fragment thereof or fusion protein binds to TMEM106B(120-254) aggregate with an affinity of 10.0 nM K_{D} or stronger.

In embodiments, the antibody or antigen-binding fragment thereof or fusion protein binds to TMEM106B(120-254) aggregate with an affinity of 2.0 nM K_{D} or stronger.

In embodiments, the antibody or antigen-binding fragment thereof or fusion protein binds to TMEM106B(120-254) aggregate with an affinity of 1.0 nM K_{D} or stronger.

In embodiments, the antibodies of the invention have an EC₅₀ for the antigen TMEM106B(120-254) aggregate of 100 ng/mL or less.

In embodiments, the antibodies of the invention specifically bind to the antigen TMEM106B(120-254) aggregate.

An antibody which specifically binds TMEM106B(120-254) aggregate. An antibody which specifically binds TMEM106B(120-254) aggregate for use in a method of treating a neurodegenerative disease. In embodiments, the neurodegenerative disease is Frontotemporal Lobar Degeneration, Lewy Body Dementia, ALS or Alzheimer's disease, comprising administering to a subject having the Frontotemporal Lobar Degeneration, Lewy Body Dementia, ALS or Alzheimer's disease.

An epitope that “specifically binds” an antibody or a polypeptide is a term well understood in the art, and methods to determine such specific or preferential binding are also well known in the art. A molecular entity is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a given sequence in TMEM106B(120-254) aggregate is an antibody that binds this epitope with greater affinity, avidity, more readily, and/or with greater duration than it binds to other epitopes. In embodiments of the antibodies or fragments, herein the antibodies or fragments preferentially bind TMEM106B(120-254) aggregate. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding.

Depending on the amino acid sequences of the constant domains of their heavy chains, antibodies (immunoglobulins) can be assigned to different classes. The antibody or fragment can be, e.g., any of an IgG, IgD, IgE, IgA or IgM antibody or fragment thereof, respectively. In an embodiment the antibody is an immunoglobulin G. In an embodiment the antibody fragment is a fragment of an immunoglobulin G. In an embodiment the antibody is an IgG1, IgG2, IgG2a, IgG2b, IgG3 or IgG4. In an embodiment the antibody comprises sequences from a human IgG1, human IgG2, human IgG2a, human IgG2b, human IgG3 or human IgG4. A combination of any of these antibody subtypes can also be used. One consideration in selecting the type of antibody to be used is the desired serum half-life of the antibody. For example, an IgG generally has a serum half-life of 23 days, IgA 6 days, IgM 5 days, IgD 3 days, and IgE 2 days. (Abbas A K, Lichtman A H, Pober J S. Cellular and Molecular Immunology, 4th edition, W. B. Saunders Co., Philadelphia, 2000, hereby incorporated by reference in its entirety).

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) (or CDRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

Also provided is a chimeric antigen receipt, or a cell expressing such, wherein the CAR comprises a heavy chain and a light chain, for example in the form of an ScFv, comprising CDRs as described herein. For example, the Vh and Vl of an scFv of the CAR can be composed of four framework regions and three CDRs described herein.

The “light chains” of antibodies (immunoglobulins) from any vertebrate species can be assigned to one of two clearly distinct types, called kappa (κ) and lambda (λ), based on the amino acid sequences of their constant domains.

“Framework” or “FR” residues are those variable domain residues other than the HVR residues as herein defined.

The term “hypervariable region” or “HVR” when used herein refers to the regions of an antibody variable domain which are hypervariable in sequence and/or form structurally defined loops. Generally, antibodies comprise six HVRs; three in the VH (H1, H2, H3) and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996). A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991) hereby incorporated by reference in its entirety) and are used herein unless otherwise specified. There are CDRs 1, 2, and 3 for each of the heavy and light chains. Chothia refers instead to the location of the structural loops (Chothia and Lesk, J. Mol. Biol. 196:901-917 (1987)). The AbM HVRs represent a compromise between the Kabat HVRs and Chothia structural loops and are used by Oxford Molecular's AbM antibody modeling software. The “contact” HVRs are based on an analysis of the available complex crystal structures. HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine of the Fc region may be removed, for example, during production or purification of the antibody, or by recombinantly engineering the nucleic acid encoding a heavy chain of the antibody. Accordingly, an intact antibody as used herein may be an antibody with or without the otherwise C-terminal lysine. In an embodiment, the Fc domain has the same sequence or 99% or greater sequence similarity with a human IgG1 Fc domain. In an embodiment, the Fc domain has the same sequence or 99% or greater sequence similarity with a human IgG2 Fc domain. In an embodiment, the Fc domain has the same sequence or 99% or greater sequence similarity with a human IgG3 Fc domain. In an embodiment, the Fc domain has the same sequence or 99% or greater sequence similarity with a human IgG4 Fc domain. In an embodiment, the Fc domain is not mutated. In an embodiment, the Fc domain is mutated at the CH2—CH3 domain interface to increase the affinity of IgG for FcRn at acidic but not neutral pH (Dall'Acqua et al, 2006; Yeung et al, 2009). In an embodiment, the Fc domain has the same sequence as a human IgG1 Fc domain.

In embodiments, the variable regions disclosed herein are not modified. In embodiments, the invention encompasses modifications to the variable regions disclosed herein. For example, the invention includes antibodies comprising functionally equivalent variable regions and CDRs which do not significantly affect their properties as well as variants which have enhanced or decreased activity and/or affinity. For example, the amino acid sequence may be mutated to obtain an antibody with the desired binding affinity to human TMEM106B(120-254). Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but framework alterations are also contemplated. Conservative substitutions are shown in Table 1 under the heading of“conservative substitutions.” If such substitutions result in a change in biological activity, then more substantial changes, denominated “exemplary substitutions” in Table 1, or as further described below in reference to amino acid classes, may be introduced and the products screened.

TABLE 1
Amino Acid Substitutions

| Original | Conservative | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

A modification or mutation may also be made in a framework region or constant region to increase the half-life of an antibody. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. According to the invention, a single antibody may have mutations in any one or more of the CDRs or framework regions of the variable domain or in the constant region.

In an embodiment, an antibody described herein is recombinantly produced. In an embodiment, the fusion protein is produced in a eukaryotic expression system.

In an embodiment, the fusion protein produced in the eukaryotic expression system comprises glycosylation at a residue on the Fc portion corresponding to Asn297.

Compositions or pharmaceutical compositions comprising the antibodies, ScFvs or fragments of antibodies disclosed herein are preferably comprise stabilizers to prevent loss of activity or structural integrity of the protein due to the effects of denaturation, oxidation or aggregation over a period of time during storage and transportation prior to use. The compositions or pharmaceutical compositions can comprise one or more of any combination of salts, surfactants, pH and tonicity agents such as sugars can contribute to overcoming aggregation problems. Where a composition or pharmaceutical composition of the present invention is used as an injection, it is desirable to have a pH value in an approximately neutral pH range, it is also advantageous to minimize surfactant levels to avoid bubbles in the formulation which are detrimental for injection into subjects. In an embodiment, the composition or pharmaceutical composition is in liquid form and stably supports high concentrations of bioactive antibody in solution and is suitable for inhalational or parenteral administration. In an embodiment, the composition or pharmaceutical composition is suitable for intravenous, intramuscular, intraperitoneal, intradermal and/or subcutaneous injection. In an embodiment, the composition or pharmaceutical composition is in liquid form and has minimized risk of bubble formation and anaphylactoid side effects. In an embodiment, the composition or pharmaceutical composition is isotonic. In an embodiment, the composition or pharmaceutical composition has a pH or 6.8 to 7.4.

In an embodiment the ScFvs or fragments of antibodies disclosed herein are lyophilized and/or freeze dried and are reconstituted for use.

Examples of pharmaceutically acceptable carriers include, but are not limited to, phosphate buffered saline solution, sterile water (including water for injection USP), emulsions such as oil/water emulsion, and various types of wetting agents. Preferred diluents for aerosol or parenteral administration are phosphate buffered saline or normal (0.9%) saline, for example 0.9% sodium chloride solution, USP. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, the content of each of which is hereby incorporated in its entirety). In non-limiting examples, this can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxy)ethoxy]ethyl (E)-octadec-9-enoate), disodium edetate dehydrate, sucrose, monobasic sodium phosphate monohydrate, and dibasic sodium phosphate dihydrate.

The antibodies, or fragments of antibodies, or compositions, or pharmaceutical compositions described herein can also be lyophilized or provided in any suitable forms including, but not limited to, injectable solutions or inhalable solutions, gel forms and tablet forms.

In an embodiment the composition or pharmaceutical composition comprising the antibody, or antigen-binding fragment thereof, described herein is substantially pure with regard to the antibody, or antigen-binding fragment thereof. A composition or pharmaceutical composition comprising the antibody, or antigen-binding fragment thereof, described herein is “substantially pure” with regard to the antibody or fragment when at least 60% to 75% of a sample of the composition or pharmaceutical composition exhibits a single species of the antibody, or antigen-binding fragment thereof. A substantially pure composition or pharmaceutical composition comprising the antibody, or antigen-binding fragment thereof, described herein can comprise, in the portion thereof which is the antibody, or antigen-binding fragment, 60%, 70%, 80% or 90% of the antibody, or antigen-binding fragment, of the single species, more usually about 95%, and preferably over 99%. Purity or homogeneity may be tested by a number of means well known in the art, such as polyacrylamide gel electrophoresis or HPLC.

Administration can be auricular, buccal, conjunctival, cutaneous, subcutaneous, endocervical, endosinusial, endotracheal, enteral, epidural, via hemodialysis, interstitial, intrabdominal, intraamniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronary, intradermal, intradiscal, intraductal, intraepidermal, intraesophagus, intragastric, intravaginal, intragingival, intraileal, intraluminal, intralesional, intralymphatic, intramedullary, intrameningeal, intramuscular, intraocular, intraovarian, intraepicardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intraventricular, intravesical, intravitreal, laryngeal, nasal, nasogastric, ophthalmic, oral, oropharyngeal, parenteral, percutaneous, periarticular, peridural, rectal, inhalationally, retrobulbar, subarachnoid, subconjuctival, sublingual, submucosal, topically, transdermal, transmucosal, transplacental, transtracheal, ureteral, uretheral, and vaginal.

In embodiments, the antibody, fragment or fusion protein is administered at a dose of 0.5 mg/kg to 100 mg/kg. In embodiments, the antibody, fragment or fusion protein is administered at a dose of 101 mg/kg to 250 mg/kg. In embodiments, the antibody, fragment or fusion protein is administered at a dose of 251 mg/kg to 500 mg/kg. In embodiments, the antibody, fragment or fusion protein is administered at a dose of 501 mg/kg to 1000 mg/kg. In embodiments, the antibody, fragment or fusion protein is administered at a dose of 1001 mg/kg to 2000 mg/kg. In embodiments, the antibody, fragment or fusion protein is administered at a dose of up to 25 mg twice per daily, daily, every other day, weekly, monthly or every three months. In embodiments, the antibody, fragment or fusion protein is administered at a dose of 25 to 100 mg twice per daily, daily, every other day, weekly, monthly or every three months. In embodiments, the antibody, fragment or fusion protein is administered at a dose of 100 to 250 mg twice per daily, daily, every other day, weekly, monthly or every three months. In embodiments, the antibody, fragment or fusion protein is administered at a dose of 250 to 500 mg twice per daily, daily, every other day, weekly, monthly or every three months. In embodiments, the antibody, fragment or fusion protein is administered at a dose of 500 to 1000 mg twice per daily, daily, every other day, weekly, monthly or every three months. In embodiments, the antibody, fragment or fusion protein is administered at a dose of 1000 to 2000 mg twice per daily, daily, every other day, weekly, monthly or every three months.

“And/or” as used herein, for example, with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

### DEFINITIONS

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means a mammal. Mammals include canines, felines, rodents, bovine, equines, porcines, ovines, and primates including humans. Thus, the invention can be used in human medicine or also in veterinary medicine, e.g., to treat companion animals, farm animals, laboratory animals in zoological parks, and animals in the wild. The invention is particularly desirable for human medical applications. In a preferred embodiment the subject is a human.

The term “patient” as used in this application means a human subject. In some embodiments of the present invention, the “patient” is one suffering with a dementia, such as Frontotemporal Lobar Degeneration, Lewy Body Dementia, ALS or Alzheimer's disease.

The terms “treat”, “treatment” of a disease, and the like refer to slowing down, relieving, ameliorating or alleviating at least one of the symptoms of the disease, or reversing the disease after its onset, preventing or reducing dementia symptoms.

The terms “prevent”, “prevention”, and the like refer to acting prior to overt disease or disorder onset, to prevent the disease or disorder from developing or to minimize the extent of the disease or disorder or slow its course of development.

The term “in need thereof” with regard to a subject would be a subject known or suspected of having or being at risk of developing a TMEM106B aggregate.

A subject in need of treatment would be one that has already developed the disease or disorder. A subject in need of prevention would be one with risk factors for Frontotemporal Lobar Degeneration, Lewy Body Dementia, ALS or Alzheimer's disease.

The terms “therapeutically effective amount” or “amount effective to” encompasses an amount sufficient to ameliorate or prevent a symptom or sign of the medical condition. Effective amount also means an amount sufficient to allow or facilitate diagnosis. An effective amount for a particular subject may vary depending on factors such as the condition being treated, the overall health of the patient, the method route and dose of administration and the severity of side effects. An effective amount can be the maximal dose or dosing protocol that avoids significant side effects or toxic effects.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

In embodiments, the antibodies or fragments as described herein, with or without detectable marker(s), can be used to quantify or measure the level of TMEM106B(120-254) in a sample or tissue as employed in methods herein described.

### Experiments—I

Generation of monoclonal antibodies to TMEM106B Fibrils

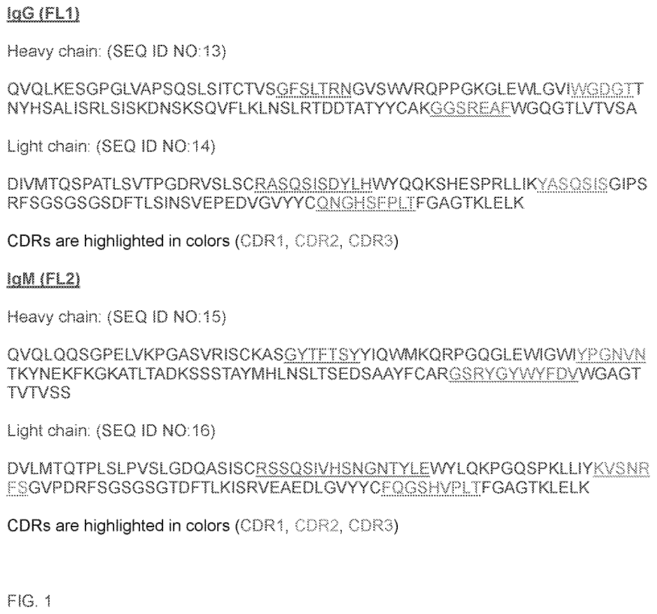

FIG. 1: Diagram of IgG sequences (VH SEQ ID NO:13; VL SEQ ID NO:14). Underlined residues are CDR1-3, consecutively; and of IgM sequences (VH SEQ ID NO:15; VL SEQ ID NO:16). Underlined residues are CDR1-3, consecutively. Antibodies were generated as described hereinbelow.

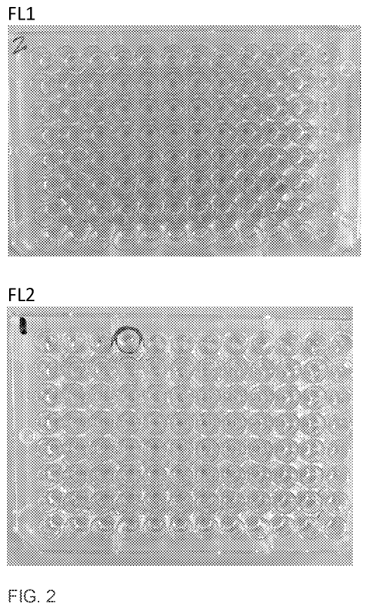

FIG. 2: Generation of monoclonal antibodies FL1 and FL2 to TMEM106B Fibrils; One mouse was immunized IP with 7.5□g of insoluble TMEM106B emulsified in freund's adjuvant on three occasions. The mouse rested 4.5 weeks, was boosted on with fibrils in PBS (w/o adjuvant), and was later sacrificed. The spleen was removed and frozen as a cell suspension in DMSO containing freezing media at −80 °C. Later, the frozen spleen cells were thawed and immediately fused with a parental NS-1 cell line grown to log phase. Supernatants from the fused cells were screened by ELISA on TMEM106B C-terminal peptide (100□L/well at 20□g/mL). FL1 and FL2 went through 2 rounds of single cell cloning. FL1 was isotyped as IgG2b and FL2 was isotyped as IgM.

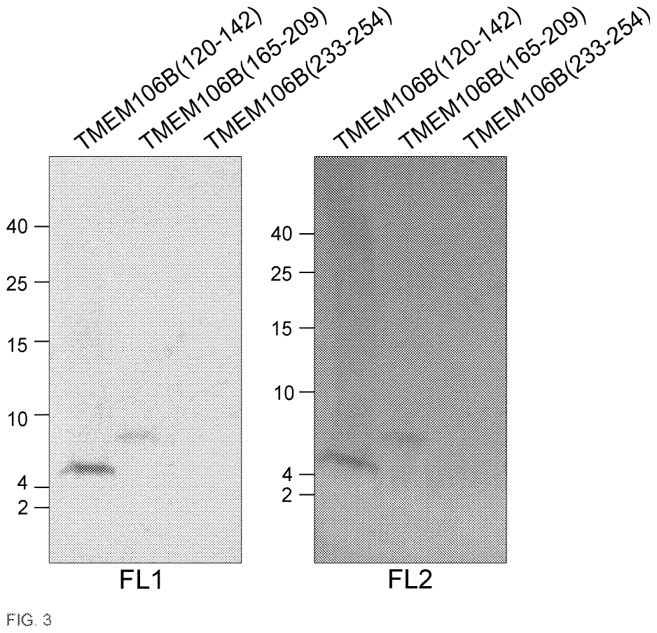

FIG. 3: Immunoblotting using TMEM106B peptides to map the epitopes of FL1 and FL2 antibodies. Results indicate that FL1 (IgG) and FL2 (IgM) bind to the N-terminal region of the fibril-forming fragment, TMEM106B(120-254). The amino acid sequence of TMEM106B(120-254) is:

(SEQ ID NO: 17)
SIDVKYIGVKSAYVSYDVQKRTIYLNITNTLNITNNNYYSVEVENITAQ
VQFSKTVIGKARLNNITIIGPLDMKQIDYTVPTVIAEEMSYMYDFCTLI
SIKVHNIVLMMQVTVTTTYFGHSEQISQERYQYVDCG.

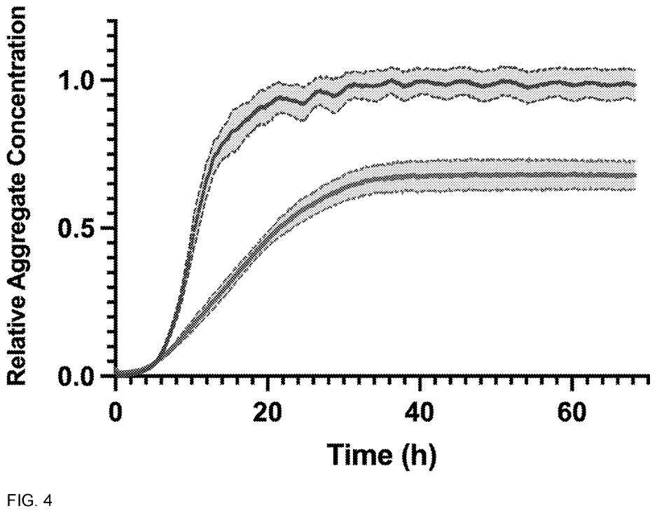

FIG. 4: Normalized Thioflavin T fluorescence intensity of an aggregation prone fragment of TMEM106B (blue curve—upper) and in the presence of FL1 antibody (red curve—lower). Protein concentration is 5 □M in 50 mM sodium phosphate, 150 mM sodium chloride, 1.25% DMSO at pH 7.4 heated to 37 °C (blue curve—upper curve) and in the presence of 1:20 molar equivalent of FL1 antibody at a concentration of 0.25 □M (red curve—lower curve). Final concentration of Thioflavin T is 40 □M. Kinetic profiles of aggregation of TMEM106B alone and in the presence of the IgG antibody. The antibody significantly slows aggregation at a sub-stoichiometric ratio (1:20 molarity).

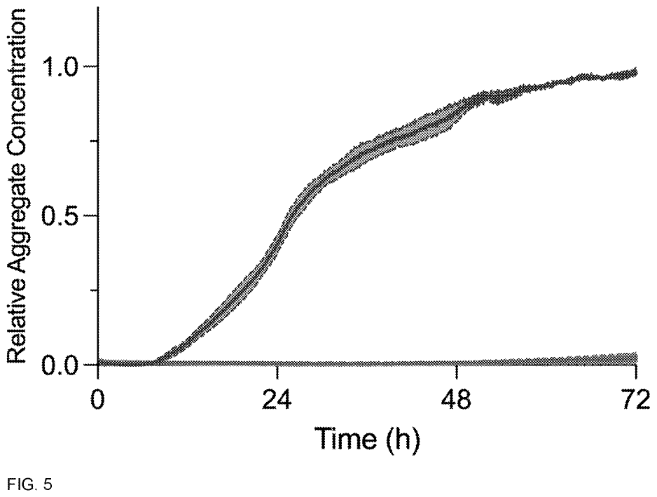

The FL2 antibody results are shown in FIG. 5 which shows Normalized Thioflavin T fluorescence intensity of an aggregation prone fragment of TMEM106B (blue curve—upper curve) and in the presence of FL2 antibody (red curve—lower curve). Protein concentration is 5 μM in 50 mM sodium phosphate, 150 mM sodium chloride, 1.25% DMSO at pH 7.4 heated to 37 °C (blue curve) and in the presence of FL1 antibody at a final concentration of 0.25 μM (red curve). Final concentration of Thioflavin T is 40 μM.

### Experiments—II

We disclose the use of TMEM106B-specific antibodies and phospho-CRMP-2 antibodies to examine human biofluids for elevated levels of hyperphosphorylated CRMP-2 in patients diagnosed with ALS and FTLD-TDP43.

As CRMP-2 can be detected in peripheral blood lymphocytes, it is informative to determine whether peripheral blood levels of CRMP-2 accurately reflect processes within the brain. If the concentration of hyperphosphorylated CRMP-2 in blood or cerebrospinal fluid (CSF) correlates with the severity of the disease, this method could serve as a rapid, minimally invasive, and highly sensitive approach to assess the effectiveness of gene therapy in clinical trials.

The utilization of phospho-CRMP-2 antibodies to assess hyperphosphorylated CRMP-2 in ALS and FTLD-TDP43 patients offers profound implications. Within the field, it can potentially revolutionize diagnostic and monitoring approaches by enabling non-invasive tracking of disease progression. Furthermore, it may streamline the evaluation of gene therapy efficacy in clinical trials. Beyond the current scope, this method has broader applications in neurodegenerative disorders and biomarker research, enabling early disease detection and treatment assessment. Its minimally invasive nature could make it accessible for widespread clinical use, benefiting patients and advancing our understanding of these challenging conditions.

Hyperphosphorylation of CRMP2 (Collapsin Response Mediator Protein-2) has been implicated in neurodegeneration. CRMP-2 is a cytosolic protein involved in various cellular processes, including neuronal development, axon guidance, and synaptic plasticity.

Hyperphosphorylation of CRMP-2 can lead to dysregulation of its normal functions and contribute to neuronal dysfunction and degeneration. One of the key kinases involved in CRMP-2 hyperphosphorylation is glycogen synthase kinase-30 (GSK-30). Increased GSK-3β activity, which can occur in neurodegenerative conditions, leads to the hyperphosphorylation of CRMP-2. This phosphorylation disrupts the interaction of CRMP-2 with microtubules and alters its role in axonal growth and synaptic function.

The dysregulation of CRMP-2 phosphorylation and its subsequent effects on neuronal processes may contribute to the pathogenesis of neurodegenerative diseases. For example, schizophrenia patients are characterized by excess nonphosphorylated (active) CRMP-2 (1). By contrast, we have found that high levels of hyperphosphorylated (inactive) CRMP-2 are present in sarkosyl-insoluble pellets fractionated from frontotemporal lobar degeneration with TDP-43 pathology (FTLD-TDP) human tissue samples. Cryoelectron microscopy (cryo-EM) was used to determine the structure of an unidentified 212 kDa protein complex to 3 Å with phosphorylation of seven residues found by mass spectrometry (MS). As a targeted-MS approach, phospho-proteomics of FTLD-TDP tissue, performed by the Fitzpatrick lab, indicates seven disease-specific phosphorylation sites (PMID: 35247328) which can be probed for in hundreds of tissue samples.

A recent study indicates that CRMP-2 protein, which is encoded by the DPYSL2 gene, may be expressed at high-levels across human spinal cord tissue with an expression profile similar to genes associated with ALS. Intriguingly, we have not observed CRMP-2 in sarkosyl-insoluble pellets fractionated from other diseased human tissue samples, e.g., Alzheimer's disease (AD), Corticobasal Degeneration (CBD), Progressive Supranuclear Palsy (PSP), though it has been detected in the sarkosyl-soluble fraction of AD tissue samples (3). This suggests that this hyperphosphorylated, soluble protein complex may be associated with protein aggregates unique to FTLD-TDP/ALS, such as filaments formed by TDP-43 (4) and TMEM106B (5), making it a suitable target for biomarker discovery. We find that TMEM106B fibrils are enriched in FTLD-TDP cases relative to neurologically normal, age-matched controls (PMID: 35247328). Progranulin, a glycoprotein deficient in frontotemporal dementia, immunoprecipitates with TMEM106B from FTLD-TDP tissue (Fitzpatrick lab, unpublished), indicating disease-specificity. We have also shown that CRMP-2 immuno-precipitates with TMEM106B from FTLD-TDP tissue in sarksoyl-insoluble fraction using a commercial antibody (ranking in the top 10% of all hits using mass spectrometry). The experimental plan is to sequentially co-IP with a phospho-TDP43 antibody followed by a TMEM106B-specific antibody as already established in the Fitzpatrick lab. phospho-TDP43 antibodies are commercially available, e.g., ThermoFisher/Invitrogen, R&D Systems, Proteintech, Cosmo Bio USA etc., including polyclonal antibodies such as Anti phospho TDP-43 (pS409/410) pAb and 22309-1-ap. This combination increases confidence in the specificity of the FTLD and ALS interactome employing mass spectrometry assays.

Using phospho-CRMP-2 antibodies to screen human biofluids is useful where there is the presence of high-levels of hyperphosphorylated CRMP-2 in ALS and FTLD-TDP43 patients. CRMP-2 is found in peripheral blood lymphocytes and is estimated to reflect intracerebral processes. To quantitatively assess the differential levels of hyper-phosphorylated CRMP-2 in sarksoyl-insoluble fraction, we will perform our immuno-assay on ALS/FTLD and other neurodegenerative diseases such as Alzheimer's and Parkinson's diseases. Where the level of hyperphosphorylated CRMP-2 in blood or cerebrospinal fluid (CSF) tracks with disease severity, this method is usable as a rapid, minimally invasive, sensitive method to measure efficacy of, for example, gene and drug therapy in clinical trials.

### REFERENCES

1. Nomoto, M., et al. (2021) Clinical evidence that a dysregulated master neural network modulator may aid in diagnosing schizophrenia. Proc Natl Acad Sci USA, 118, e2100032118.
2. Yadav A, et al. (2023) A cellular taxonomy of the adult human spinal cord. Neuron, 111, 328-344.e7.
3. Cole, A. R., et al. (2007) Collapsin response mediator protein-2 hyperphosphorylation is an early event in Alzheimer's disease progression. J Neurochem, 103, 1132-1144.
4. Arseni, D., et al. (2022) Structure of pathological TDP-43 filaments from ALS with FTLD. Nature, 601, 139-143.
5. Chang, A., et al. (2022) Homotypic fibrillization of TMEM106B across diverse neurodegenerative diseases. Cell, 185, 1346-1355.e15.

1. An antibody, antigen-binding fragment thereof, or fusion protein, which binds TMEM106B(120-254) aggregate, comprising:
a)
a heavy chain comprising:
CDR1 having the sequence
(SEQ ID NO: 1)
GFSLTRN;
CDR2 having the sequence
(SEQ ID NO: 2)
WGDGT;
CDR3 having the sequence
(SEQ ID NO: 3)
GGSREAF;
and
a light chain comprising:
CDR1 having the sequence
(SEQ ID NO: 4)
RASQSISDYLH;
CDR2 having the sequence
(SEQ ID NO: 5)
YASQSIS;
CDR3 having the sequence
(SEQ ID NO: 6)
QNGHSFPLT;
or
b)
a heavy chain comprising:
CDR1 having the sequence
(SEQ ID NO: 7)
GYTFTSY;
CDR2 having the sequence
(SEQ ID NO: 8)
YPGNVN;
CDR3 having the sequence
(SEQ ID NO: 9)
GSRYGYWYFDV;
and
a light chain comprising:
CDR1 having the sequence
(SEQ ID NO: 10)
RSSQSIVHSNGNTYLE;
CDR2 having the sequence
(SEQ ID NO: 11)
KVSNRFS;
CDR3 having the sequence
(SEQ ID NO: 12)
FQGSHVPLT.

2. The antibody, fragment thereof, or fusion protein of claim 1, wherein the heavy chain comprises:
CDR1 having the sequence
(SEQ ID NO: 1)
GFSLTRN;
CDR2 having the sequence
(SEQ ID NO: 2)
WGDGT;
CDR3 having the sequence
(SEQ ID NO: 3)
GGSREAF;
and the light chain comprises:
CDR1 having the sequence
(SEQ ID NO: 4)
RASQSISDYLH;
CDR2 having the sequence
(SEQ ID NO: 5)
YASQSIS;
CDR3 having the sequence
(SEQ ID NO: 6)
QNGHSFPLT.

3. The antibody, fragment thereof, or fusion protein of claim 1, wherein the heavy chain comprises:
CDR1 having the sequence
(SEQ ID NO: 7)
GYTFTSY;
CDR2 having the sequence
(SEQ ID NO: 8)
YPGNVN;
CDR3 having the sequence
(SEQ ID NO: 9)
GSRYGYWYFDV;
and the light chain comprises:
CDR1 having the sequence
(SEQ ID NO: 10)
RSSQSIVHSNGNTYLE;
CDR2 having the sequence
(SEQ ID NO: 11)
KVSNRFS;
CDR3 having the sequence
(SEQ ID NO: 12)
FQGSHVPLT.

4. The antibody, fragment thereof, or fusion protein of claim 1, wherein the antibody is a monoclonal antibody.

5. The antibody, fragment thereof, or fusion protein of claim 1, comprising framework regions of a light chain and/or a heavy chain which are human framework regions, or have 85% or more sequence identity thereto.

6. The antibody, fragment thereof, or fusion protein of claim 1, wherein the framework regions of the light chain and/or the heavy chain are human framework regions.

7. The antibody, fragment thereof, or fusion protein of claim 1, wherein the antibody is a humanized antibody.

8. The antibody, fragment thereof, or fusion protein of claim 1, wherein the antibody or antigen-binding fragment thereof has a human sequence Fc region.

9. The antibody, fragment thereof, or fusion protein of claim 1, wherein a heavy chain of the antibody comprises
(SEQ ID NO: 13)
QVQLKESGPGLVAPSQSLSITCTVSGFSLTRNGVSWVRQPPGKGLEWLG
VIWGDGTTNYHSALISRLSISKDNSKSQVFLKLNSLRTDDTATYYCAKG
GSREAFWGQGTLVTVSA.

10. The antibody, fragment thereof, or fusion protein of claim 1, wherein a heavy chain of the antibody comprises
(SEQ ID NO: 15)
QVQLQQSGPELVKPGASVRISCKASGYTFTSYYIQWMKQRPGQGLEWIG
WIYPGNVNTKYNEKFKGKATLTADKSSSTAYMHLNSLTSEDSAAYFCAR
GSRYGYWYFDVWGAGTTVTVSS.

11. The antibody, fragment thereof, or fusion protein of claim 1, wherein a light chain of the antibody comprises
(SEQ ID NO: 14)
DIVMTQSPATLSVTPGDRVSLSCRASQSISDYLHWYQQKSHESPRLLIK
YASQSISGIPSRFSGSGSGSDFTLSINSVEPEDVGVYYCQNGHSFPLTF
GAGTKLELK.

12. The antibody, fragment thereof, or fusion protein of claim 1, wherein a light chain of the antibody comprises
(SEQ ID NO: 16)
DVLMTQTPLSLPVSLGDQASISCRSSQSIVHSNGNTYLEWYLQKPGQSP
KLLIYKVSNRFSGVPDRFSGSGSGTDFTLKISRVEAEDLGVYYCFQGSH
VPLTFGAGTKLELK.

13. The antibody of claim 1, wherein the antibody is an IgG.

14. The antibody of claim 1, wherein the antibody is an IgM.

15. A pharmaceutical composition comprising the antibody, antigen-binding fragment thereof, or fusion protein, which binds TMEM106B(120-254) aggregate of claim 1 and a carrier.

16. A method of treating or reducing development of Frontotemporal Lobar Degeneration, Lewy Body Dementia, ALS or Alzheimer's disease, comprising administering to a subject having the Frontotemporal Lobar Degeneration, Lewy Body Dementia, ALS or Alzheimer's disease an amount of an antibody which binds TMEM106B(120-254) aggregate, or TMEM106B(120-254) aggregate binding fragment thereof or fusion protein as set forth in claim 1, effective to reduce development of or treat Frontotemporal Lobar Degeneration, Lewy Body Dementia, ALS or Alzheimer's disease.

17. A method of treating or reducing development of a neurodegenerative disorder, comprising administering to a subject having the neurodegenerative disorder an amount of an antibody which binds TMEM106B(120-254) aggregate, or TMEM106B(120-254) aggregate binding fragment thereof or fusion protein as set forth in claim 1, effective to reduce development of or treat a neurodegenerative disorder.

18. A method of reducing development of, or accumulation of, a TMEM106B(120-254) aggregate, comprising administering to a subject having a TMEM106B(120-254) aggregate or susceptible thereto an amount of an antibody which binds TMEM106B(120-254) aggregate, or TMEM106B(120-254) aggregate binding fragment thereof or fusion protein as set forth in claim 1, effective to reduce development of, or accumulation of, a TMEM106B(120-254) aggregate.

19. The method of claim 18, further comprising receiving identification of the subject, or identifying the subject, as having, or being susceptible to, a TMEM106B(120-254) aggregate prior to administration of the treatment.

20. A nucleic acid encoding a heavy chain, or a light chain, or both, as set forth in claim 1.
